(19) United States
(12) Patent Application Publication
(10) Pub. No.: US 2018/0228502 A1
(43) Pub. Date: Aug. 16, 2018

(54) DUAL LUMEN HYPOTUBE CATHETER

(71) Applicant: Penumbra, Inc., Alameda, CA (US)

(72) Inventors: Robyn Shaffer, Alameda, CA (US); Holly Sit, Alameda, CA (US)

(73) Assignee: Penumbra, Inc., Alameda, CA (US)

(21) Appl. No.: 15/892,732

(22) Filed: Feb. 9, 2018

Related U.S. Application Data
(60) Provisional application No. 62/458,339, filed on Feb. 13, 2017.

(51) Int. Cl. A61B 17/22

(52) U.S. Cl. CPC A61B 17/22; A61M 2025/0183

(57) ABSTRACT

A catheter includes a shaft having a proximal end, a distal end, and a central lumen extending continuously from the proximal end to the distal end. A hypotube forms a proximal region of the shaft, and a polymer tube forms a distal region of the shaft. A periphery of the hypotube is cut in a pattern which provides a decreasing stiffness in a proximal-to-distal direction, and in some embodiments the polymer tube is reinforced by a wire embedded over at least a portion of the polymer tube's length. A guidewire lumen is disposed at or near the distal end of the catheter and extends for a portion of the catheter's length. The catheter may be used for aspiration, device delivery, or other purposes in a patient's vasculature.

### CROSS-REFERENCE TO RELATED APPLICATION

This patent application claims the benefit of provisional patent application No. 62/458,339 (Attorney Docket No. 41507-724.101), filed on Feb. 13, 2017, the full disclosure of which is incorporated by reference.

### BACKGROUND OF THE INVENTION

### 1. Field of the Invention

The present invention relates generally to medical apparatus and medical treatment methods. More particularly, the present invention relates to the design and use of clot aspiration catheters for use in the coronary, peripheral, and neuro vasculature.

In most interventional neurology and other endovascular procedures, efficiency is a primary concern. Such interventions need to be performed quickly. Many prior devices, however, are less efficient than would be desirable. For example, some prior thrombus extraction catheters use a stylet for structural support during advancement to the target vasculature. The stylet is typically a ground metal wire that is placed within the catheter's lumen prior to insertion and advancement and prevents the catheter from kinking when traversing tortuous vasculature. As the stylet occupies the catheter's main lumen, the stylet must be removed before aspiration can begin or devices can be delivered. Such removal takes time and delays start of the desired therapy.

Some prior art catheters incorporate a cross-hatched braided structure for catheter wall support. Such braided constructions can be difficult to fabricate and can require a thicker catheter wall which decreases inner diameter. A smaller inner diameter negatively affects aspiration performance. Possible other disadvantages of excluding a stylet include a less robust catheter that is less pushable and more susceptible to kinking.

For these reasons, it would be desirable to provide alternative and improved catheter designs and methods of use for thrombus extraction, device delivery and other purposes. It would be particularly desirable to provide a catheter that is pushable without a stylet and maximizes inner diameter through the use of thin catheter walls. The present invention meets at least some of these needs.

### 2. Description of the Background Art

Relevant patents and published applications include: U.S. Pat. No. 9,820,761; U.S. Pat. No. 8,509,916; U.S. Pat. No. 4,692,141; U.S. Pat. No. 6,945,956; U.S. Pat. No. 6,893,417; U.S. Pat. No. 9,636,477; U.S. Pat. No. 6,723,084; U.S. Pat. No. 9,078,682; U.S. Pat. No. 7,300,430; U.S. Pat. No. 9,579,485; U.S. Pat. No. 9,662,129; U.S. Pat. No. 9,532,792; U.S. Pat. No. 7,309,334; U.S. Pat. No. 6,152,909; and EP711574A1.

### SUMMARY OF THE INVENTION

The present invention provides a rapid exchange aspiration catheter that simplifies a variety of vascular procedures, including balloon angioplasty, stent delivery, device delivery, and in particular thrombectomy procedures in the coronary, peripheral, and neuro vasculature. Catheters according to the present invention can effectively reach desired treatment zones and deliver devices to the treatment zones and/or remove thrombus to re-vascularize vessels to restore normal flow.

The rapid exchange aspiration catheters of the present invention are particularly suitable for introduction to a target site immediately following placement of a guidewire. The aspiration catheters may be directly advanced over the guidewire to a target site and can aspirate thrombus and/or deliver other devices immediately following placement without the need to withdraw the guidewire. In contrast, many prior devices require the physician to remove a stabilizing stylet, guidewire, or perform other steps before the catheter is ready for aspiration or other uses. While the resulting delays may seem minor, even very short delays in commencing treatment can be critical in cases of occlusive stroke and other endovascular procedures where time is of the essence.

In addition to efficient introduction, the catheters of the present invention preferably utilize a slit or slotted hypotube in at least a proximal shaft portion, providing a controlled, typically variable, stiffness to eliminate the need for a stylet or other stiffening member for introduction. In particular, the slit or slotted hypotube may be formed by laser cutting an intact tube and may have either or both a variable cut pitch and a variable cut density (ratio of cut arc to uncut arc where cut arc is the portion of the hypotube circumference which is cut) to provide a desired variable flexibility pattern, typically with lower stiffness (higher flexibility) in the distal regions of the hypotube near the distal tip of the catheter. In specific instances, the slit or slotted hypotube may have two, three, or more distinct regions each with a differing cut pattern and local stiffness value.

In other embodiments, the catheter shaft further comprises a polymer coating over at least a portion of its outer surface, typically over the entire length of the catheter shaft, where the polymer may at least in part form a polymer wall or polymer wall component. In still other embodiments, a distal region of the catheter shaft may be free of the hypotube and incorporate an alternative support, for example a wire support, typically embedded in a polymer wall or wall component, which provides high hoop strength with a very low stiffness (high flexibility). This wire support may be helical, braided, coiled, or of an alternative suitable configuration for conferring support to the polymer wall. The combination of a proximal cut hypotube and a distal embedded wire reinforcement component in a polymer wall or coating achieves an ideal balance between rigidity and flexibility over the length of the catheter shaft. In general, the proximal portion of the shaft is relatively rigid, and the shaft becomes progressively more flexible towards its distal tip. This balance of rigidity and flexibility, in turn, provides enhanced trackability and pushability (i.e. robustness) absent from other catheter systems. Often, the distal-most tip of the catheter shaft will be free from metal or other rigid reinforcement so that it will relatively soft to provide an atraumatic tip to safely traverse delicate vasculature.

The distal tips of catheters of the present invention will usually be beveled, i.e. have a generally planar opening disposed at an angle from 20° to 80°, usually from 25° to 55°, ideally at about 30°, relative to the longitudinal axis of the catheter shaft at the tip. Too shallow of a bevel angle can inhibit the tip from engaging and sealing with clot over the entire circumference of the opening which adversely affects aspiration and can cause excess blood to be aspirated. Additionally, too shallow of an angle increases the risk of the catheter suction “cupping” onto the vessel wall and becoming anchored in place. The preferred 30° angle is shallow enough to supply a strong force on the clot, while also ensuring that the catheter tip is able to form a seal with the clot, which avoids needless blood aspiration.

In a first particular aspect, the present invention provides a catheter, typically a vascular catheter suitable for the aspiration of thrombus and/or for other purposes including device delivery, e.g. the delivery and release of coils, stents, and the like. The catheter comprises a shaft having a proximal end, a distal end, and a central lumen extending continuously from the proximal end to the distal end. A hypotube forms a proximal region of the shaft, and a polymer cover or body is formed over the hypotube and extends distally of the shaft, typically defining an unreinforced, atraumatic distal tip. A periphery of the hypotube may be cut in a pattern selected to provide a decreasing stiffness in a proximal-to-distal direction.

In specific embodiments, the catheter includes a guidewire tube having a guidewire lumen extending over a distal portion of the shaft. The guidewire tube has a distal tip and a proximal port, and the guidewire lumen is disposed in the guidewire tube and extends from the distal tip to the proximal port. The guidewire tube is usually attached to the shaft so that the guidewire lumen is parallel to and laterally offset from the central lumen. The distal tip of the guidewire tube often extends distally beyond a distal opening of the central lumen, typically by a distance in the range from 0.5 mm to 3 mm. The proximal port of the guidewire tube is typically located distally of the proximal end of the shaft to form a rapid exchange structure.

In other specific embodiments of the catheter, the shaft has a length in the range from 65 cm to 165 cm and the proximal port of the guidewire tube is located proximally of the distal end of the shaft by a distance in the range from 5 cm to 80 cm. Usually, a distal end of the hypotube extends to within 5 mm of the distal end of the shaft and the portion of the polymer body distal to a distal end of the hypotube is free from reinforcement other than a radiopaque marker ring. In other instances, a distal end of the hypotube extends to more than 5 mm from the distal end of the shaft and the portion of the polymer body distal to a distal end of the hypotube is reinforced with a wire. Typically, the wire support is configured in a manner that will confer gradually decreasing stiffness in a proximal to distal direction. In the example of a helical wire, a pitch of the helical wire increases (i.e. the spacing between adjacent turns of the coil increases) in a proximal-to-distal direction to provide a decreasing stiffness.

In still other specific embodiments of the catheter, the polymer tube has a beveled end. In specific instances, the polymer tube has a generally planar opening disposed at an angle from 20° to 80° relative to the longitudinal axis of the catheter shaft at the distal end. In other specific instances, a distal tip or region of the polymer tube is free from reinforcement from the wire reinforcement in order to form an atraumatic tip. Usually, the catheter shaft further comprises a radiopaque marker near the distal end of the shaft and optional length indicative markers on the proximal end of the shaft.

In a second particular aspect, the present invention provides a method for aspirating thrombus. The method comprises providing a catheter having a structure as described above. A distal end of a shaft of the catheter is advanced to a region in the vasculature at least partially occluded by thrombus. A distal tip of the catheter is engaged against the thrombus to create a seal, and a negative pressure is applied to a proximal end of a central lumen of the shaft to aspirate thrombus through the central lumen.

In specific embodiments of the methods, the catheter is advanced over a guidewire, and catheter is advanced through a guide catheter. The distal end of the shaft is usually beveled to enhance the seal formed with the thrombus and the vasculature is typically selected from the group consisting of coronary, peripheral, and neuro vasculature.

### BRIEF DESCRIPTION OF DRAWINGS

FIG. 1 illustrates an exemplary aspiration catheter constructed in accordance with the principles of the present invention.

FIG. 1A is a longitudinal cross-sectional view of the distal end of a first embodiment of the catheter of FIG. 1 taken within region 1A-1A and showing a hypotube reinforcement extending to a location just proximal of the distal end of the catheter shaft.

FIG. 1B is a lateral cross-sectional view taken along line 1B-1B of FIG. 1.

FIG. 1C is a lateral cross-sectional view taken along line 1C-1C of FIG. 1.

FIG. 2 illustrates an exemplary embodiment of hypotube structure that can be used in the proximal shaft portion of the catheter of FIG. 1.

FIG. 3 an extended longitudinal cross-sectional view of an alternative embodiment of a distal portion of the catheter of FIG. 1 showing a distal region comprising a wire-reinforced polymer tube.

### DETAILED DESCRIPTION OF THE SPECIFIC EMBODIMENTS

The present invention provides a dual lumen clot aspiration catheter having a main lumen and a discrete length of guidewire lumen that, when stacked, form an asymmetric cross-section. Previous catheters had dual lumens but typically required a stylet to improve pushability and to provide kink resistance. The stylet was typically a round wire with proximal stiffness and distal flexibility. To maintain pushability and kink resistance without a stylet, other catheters utilize a thicker braided catheter wall design that reduces inner diameter potential. Examples of catheters with one or more of these shortcomings include the Medtronic Export™ Advance Aspiration Catheter and the Vascular Solutions Pronto® Extraction Catheter.

The catheters of the present invention comprise a slit or slotted hypotube over at least a proximal portion of a catheter shaft to provide pushability and kink resistance without a stylet while retaining flexibility and maximizing inner diameter. In some embodiments, the hypotube extends the entire length of the catheter shaft. In other embodiments, the hypotube is present only on a proximal region of the catheter shaft. Optionally, a distal portion of the catheter shaft may comprise a wire-reinforced polymer tube, usually a helical wire reinforcement, to provide hoop strength at the distal tip where negative pressure is applied to aspirate thrombus present in the target vasculature. A guide wire lumen is typically provided over the distal portion of the catheter shaft, usually from 5 cm to 80 cm, typically over the distal-most 30 cm of the device.

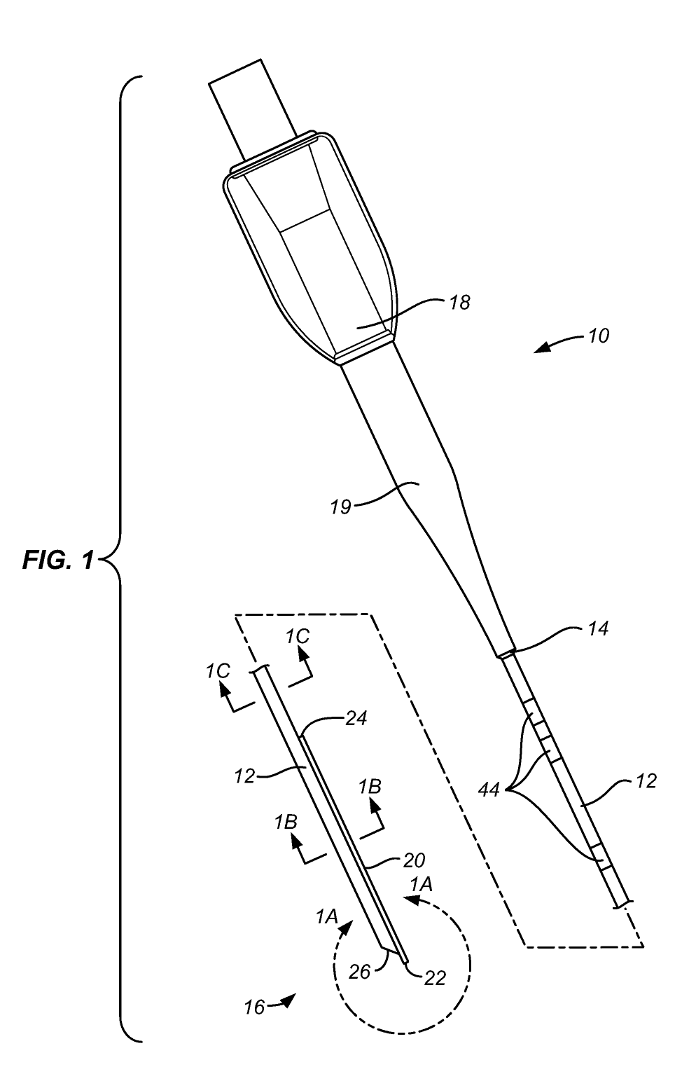

As shown in FIGS. 1, 1A, 1B and 1C, a rapid exchange catheter 10 constructed in accordance with the principles of the present invention comprises a shaft 12 having a proximal end 14, a distal end 16, and a central lumen 28 extending from the proximal end to the distal end for an effective length typically between 65 cm to 165 cm. A hub 18 is attached to the proximal end of the shaft and comprises a conventional winged structure having a strain relief portion 19 which is secured to a hypotube 34 disposed in the shaft 12, as described in greater detail below. A guidewire tube 20 is secured to a distal region of the shaft 12 and has a distal tip 22 and a proximal port 24. The guidewire tube and lumen is used for advancement of the catheter over a guidewire in the vasculature, typically through a guide catheter in most procedures. The total length of the guidewire tube and lumen is typical in a range from 8 cm to 80 cm, usually being around 30 cm. A distal portion of shaft of the catheter will have usually have a lubricious and durable hydrophilic coating formed over the exterior surface in order to improve the catheter's trackability through tortuous vasculature. Typically, the central lumen 28 will be coated with a polymer 36. Optionally, a plurality of length indicators 44 are positioned over a proximal portion of the shaft 12 and a single distal radiopaque marker 42 is positioned at or near the distal end of the shaft. The radiopaque marker is typically a platinum or other metal ring which extends partially or fully circumferentially about the shaft and which is used to observe and position the catheter under fluoroscopy.

In a first embodiment, as shown specifically in FIGS. 1A, 1B and 1C, the hypotube 34 extends from the hub 18 to a location just proximal of the distal end 16 of the catheter shaft 12. See FIG. 1A. The guidewire tube 20 sits atop the distal end of the catheter shaft 12 and extends slightly beyond the aspiration catheter shaft's distal end, typically by a distance d₁ in the range from 0.5 mm to 3 mm, usually from 1.0 mm to 1.5 mm. Such distal extension of the distal tip 22 of the guidewire tube 20 is advantageous because it increases the ease of inserting the proximal end of the guidewire into the distal end of the guidewire lumen by the user.

As further shown in FIG. 1A, the distal end 16 of the catheter shaft 12 is usually beveled, i.e. the distal opening 26 of the lumen 28 is formed in a generally planar face that is inclined at an angle α in the range from 20° to 80°, typically being about 30°, relative to a longitudinal axis of the catheter shaft at the distal end. Such a bevel optimizes the balance between creating a seal, which promotes a strong suction force for aspiration while also preserving ease of trackability.

FIG. 1B is a cross-sectional view of the shaft 12 at a location where guidewire tube 20 is attached to the main shaft body. The lumens 28 and 30 independently have circular cross-sections. However, when stacked they create an asymmetric cross-section. Since the guidewire lumen is smaller than the hypotube lumen, their combination in a composite forms an overall tear-drop shaped cross-section. The guidewire lumen 30 at the top has an inner diameter d₂ typically in a range from about 0.4 mm to 1.3 mm, usually being about 0.4 mm to 1 mm, often being 0.4 mm. This sizing enables the guidewire lumen to accept standard guidewires. FIG. 1B also shows an exemplary maximum profile or width W₁ in range from about 1 mm to about 5 mm, usually being about 1.5 mm to 2 mm, often being 1.75 mm. The central lumen d₃ typically has a diameter in a range from about 0.5 mm to 2.5 mm, usually being about 1 mm to 1.5 mm, often being 1.1 mm. These dimensions allow the catheter to fit within standard guide catheters.

FIG. 1C is a cross-sectional view of the shaft 12 at a location proximal of the port 24 of the guidewire tube 20. Aspiration lumen 28 in the hypotube 34 of the shaft 12 is the only remaining lumen at this point. As the shaft no longer accommodates the guidewire tube 20, the overall outer diameter is smaller, typically to a width W₂ in a range from 0.9 mm to 3.1 mm, often being 1.4 mm. The inner diameter d₃ of the central lumen remains constant over the entire length of the catheter shaft.

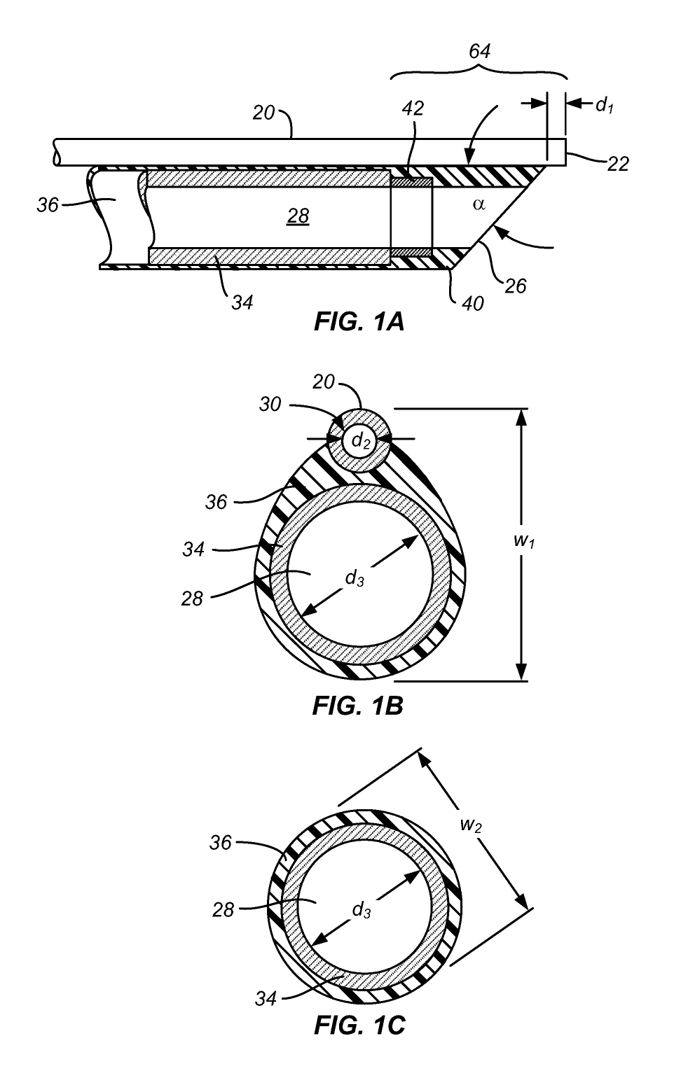

FIG. 2 illustrates an exemplary embodiment of laser cut patterns on the hypotube 34. The hypotube 34 is preferably comprises a metal such as stainless steel or a nickel-titanium alloy, e.g. a Nitinol® alloy. The hypotube will typically have a laser cut pattern 48, for example of interrupted or uninterrupted spiral cuts. The cut density (ratio of cut angle to uncut angle of the hypotube 34) and pitch variation (distance between adjacent turns of the spiral cut) of the laser cuts determines the local flexibility of the hypotube over its length. Where cut density is high and pitch distance is low, the hypotube is at its most flexible. The catheter shaft 12 typically has at least three material transition zones, each with a different stiffness and usually decreasing in the proximal-to-distal direction. Three zones in the hypotube provide proximal stiffness, distal softness, and a mid-shaft region which provides a smooth transition between the end regions.

The hypotube will typically have a cut pattern design that transitions from a flexible distal end to a stiffer proximal end. This is achieved through changes in the cut density and pitch variation. Sections of the hypotube from the distal to the proximal end can keep the two primary variables constant, vary specific variables, or transition from one set of variables to others. All of these variables are used in conjunction to create a desired stiffness profile and smooth transition through the hypotube in order to achieve tracking to the target site while optimizing device performance and robustness. As illustrated in FIG. 2, exemplary hypotube 34 has four zones 50a through 50d. The proximal-most zone 50a is free from cuts and will usually be anchored to the hub 18. The distal-most zone 50d has a constant cut density whose pitch transitions from a larger proximal pitch to a smaller distal pitch. Zone 50c has a lower cut density to increase stiffness and the pitch transitions from a smaller to larger pitch. Zone 50b has the same cut density zone 50c but a larger pitch that remains constant for its length. In section 50a, the hypotube is uncut for maximum stiffness. Another exemplary hypotube has one zone where cut density is constant and pitch transitions from a larger proximal pitch to a smaller distal pitch. A third exemplary hypotube has one zone where pitch is constant and cut density transitions from a smaller proximal density to a larger distal density. In the embodiment of FIGS. 1A to 1C, the distal tip the hypotube is flexible enough to form an atraumatic distal tip of the catheter. The hypotube 34 typically has an inner diameter of the hypotube in a range from 0.5 mm to 2.5 mm, typically being 1.1 mm, and an outer diameter in a range from 0.9 mm to 3.2 mm, typically being about 1.3 mm.

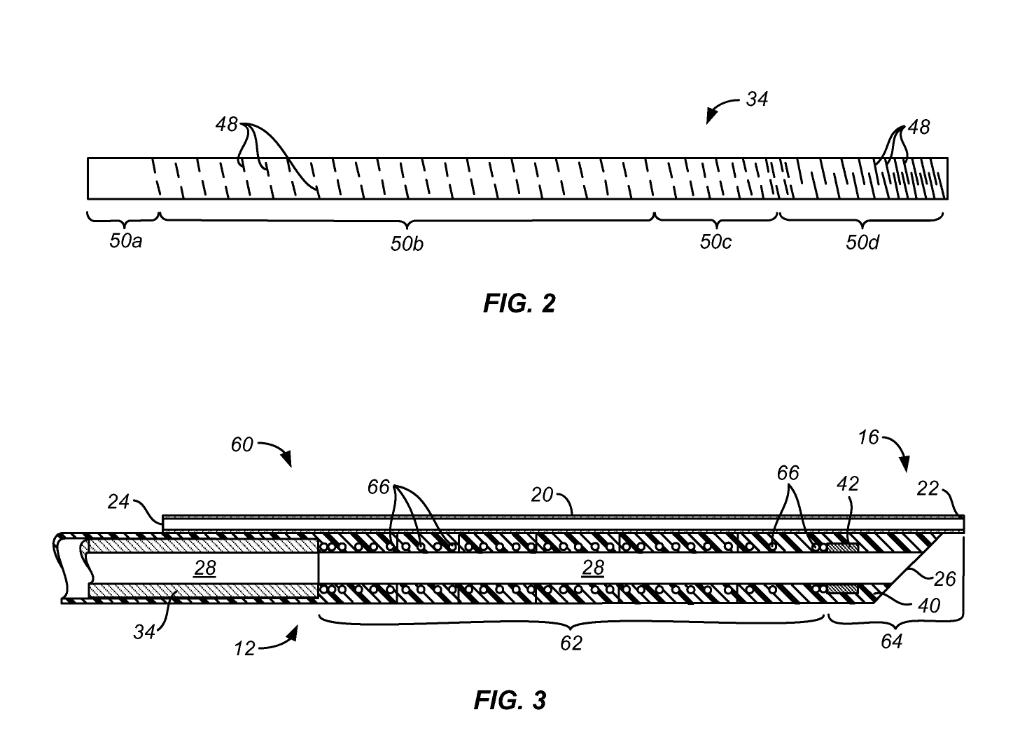

Referring to FIG. 3, an alternative embodiment 60 of the catheter of the present invention includes a wire-reinforced transition region 62 between a distal end of the hypotube 34 and an unreinforced region 64 at the atraumatic distal end 16 of the catheter shaft 12. The transition regions typically comprises a polymer body having an embedded wire reinforcement 66, allowing the distal portion of the hypotube 34 to be less flexible (more stiff). The catheter typically includes a polymer coating 36. While many features of the first catheter embodiment 10 are carried over into the second catheter embodiment 60, such as the unreinforced beveled tip and separate guide wire tube 20 having an axially extended distal tip 22, the wire-reinforced transition region 62 is unique and typically formed with a helical wire, usually a metal wire formed from a stainless steel or a nickel-titanium alloy, e.g. a Nitinol® alloy, by winding of the wire around a mandrel. After the wire reinforcement is helically or otherwise wound, the wire reinforcement is encased in a polymer body, forming both the wire-reinforced portion 62 and the unreinforced distal tip portion 64. The guidewire tube 20 typically overlaps and stabilizes the transition between and the hypotube 34 and the wire-reinforced portion 62. Other aspects of catheter 60 are generally the same as described previously with respect to catheter 10.

While the present invention has been described herein with respect to the exemplary embodiments and the best mode for practicing the invention, it will be apparent to one of ordinary skill in the art that many modifications, improvements and sub-combinations of the various embodiments, adaptations and variations can be made to the invention without departing from the spirit and scope thereof.

What is claimed is:

1. A catheter comprising:
a shaft having a proximal end, a distal end, and a central lumen extending continuously from the proximal end to the distal end;
a hypotube forming a proximal region of the shaft; and
a polymer body formed over the hypotube and extending distally of the shaft;
wherein a periphery of the hypotube is cut in a pattern selected to provide a decreasing stiffness in a proximal-to-distal direction.

2. The catheter of claim 1, wherein the shaft has a guidewire lumen extending over a distal portion of the shaft.

3. The catheter of claim 2, wherein the shaft further comprises a guidewire tube having a distal tip and a proximal port, wherein the guidewire lumen is disposed in the guidewire tube and extends from the distal tip to the proximal port.

4. The catheter of claim 3, wherein the guidewire tube is attached to the shaft so that the guidewire lumen is parallel to and laterally offset from the central lumen.

5. The catheter of claim 3, wherein the distal tip of the guidewire tube extends distally beyond a distal opening of the central lumen by a distance in the range from 0.5 mm to 3 mm.

6. The catheter of claim 3, wherein the proximal port of the guidewire tube is located distally of the proximal end of the shaft to form a rapid exchange structure.

7. The catheter of claim 6, wherein the shaft has a length in the range from 65 cm to 165 cm and the proximal port of the guidewire tube is located proximally of the distal end of the shaft by a distance in the range from 5 cm to 80 cm.

8. The catheter of claim 1, wherein a distal end of the hypotube extends to within 5 mm of the distal end of the shaft and the portion of the polymer body distal to a distal end of the hypotube is free from reinforcement other than a radiopaque markerband.

9. The catheter of claim 1, wherein a distal end of the hypotube extends to more than 5 mm from the distal end of the shaft and the portion of the polymer body distal to a distal end of the hypotube is reinforced with a wire.

10. The catheter of claim 9, wherein the wire is configured in a manner to confer a stiffness to the device that decreases in a proximal-to-distal direction.

11. The catheter of claim 1, wherein the hypotube has a variable pitch and a consistent cut density to provide a decreasing stiffness in a proximal-to-distal direction.

12. The catheter of claim 1, wherein the hypotube has a variable cut density and a consistent pitch to provide a decreasing stiffness in a proximal-to-distal direction.

13. The catheter of claim 1, wherein the hypotube has variable cut density and a variable pitch to provide a decreasing stiffness in a proximal-to-distal direction.

14. The catheter of claim 1, wherein the polymer tube has a beveled end.

15. The catheter of claim 14, wherein the polymer tube has a generally planar opening disposed at an angle from 20° to 80° relative to the longitudinal axis of the catheter shaft at the distal end.

16. The catheter of claim 9, wherein a distal tip of the polymer tube is free from reinforcement from the wire.

17. The catheter of claim 1, further comprising a radiopaque marker near the distal end of the shaft.

18. A method for aspirating thrombus, said method comprising:
providing a catheter as in claim 1;
advancing a distal end of the catheter to a region in a vasculature at least partially occluded by thrombus;
engaging a distal tip of the catheter against the thrombus to create a seal; and
applying a negative pressure to the proximal end of a central lumen of the shaft to aspirate thrombus through the central lumen.

19. A method as in claim 18, wherein the catheter is advanced over a guidewire.

20. A method as in claim 19, wherein the catheter is advanced through a guide catheter.

21. A method as in claim 18, wherein the distal end of the shaft is beveled to enhance the seal formed with the thrombus.

22. A method as in claim 18, wherein the vasculature is selected from the group consisting of coronary, peripheral, and neuro vasculature.

23. A method as in claim 18, wherein the vasculature comprises coronary vasculature.
